(19) United States
(12) Patent Application Publication
(10) Pub. No.: US 2018/0355302 A1
(43) Pub. Date: Dec. 13, 2018

(54) FERMENTER FOR PRODUCING A PSEUDOPLASTIC MEDIUM

(71) Applicant: Wintershall Holding GmbH, Kassel (DE)

(72) Inventors: Alexander SCHOLZ, Mutterstadt (DE); Florian LEHR, Schwegenheim (DE); Sascha ROLLIE, Mannheim (DE); Christian FLECK, Sandhausen (DE); Julia HOFINGER, Ludwigshafen (DE); Nadja POLLMER, Bissersheim (DE); Christian DIENES, Wernersberg (DE); Michael SCHREIBER, Mannheim (DE)

(73) Assignee: Wintershall Holding GmbH, Kassel (DE)

(21) Appl. No.: 15/573,343

(22) PCT Filed: May 10, 2016
(86) PCT No.: PCT/EP2016/060433

§ 371 Date: Nov. 10, 2017

(30) Foreign Application Priority Data
May 11, 2015 (EP) 15167132.8

(51) Int. Cl. C12M 1/06; C12M 1/02; C12P 19/04; B01F 7/00; B01F 7/22; B01F 3/04; B01F 15/00; B01F 15/06

(52) U.S. Cl. CPC C12M 27/02; C12M 41/24; C12P 19/04; B01F 7/00133; B01F 7/00141; B01F 7/00341; B01F 2215/0073; B01F 7/22; B01F 3/04602; B01F 15/00915; B01F 15/066; B01F 2003/04673; B01F 2215/0422; B01F 7/00633

(57) ABSTRACT

Fermenter for producing a shear-thinning medium comprising a tank volume and a stirring arrangement having an improved distribution capacity or a more uniform shear stress.

### FIELD OF THE INVENTION

The present invention relates to a device for the fermentation of a broth for the production of a shear-thinning medium, more particularly a fermenter for the production of polysaccharides or glucans, which fermenter allows, for the mixing of the shear-thinning medium, a uniform shear influence or a large region of low viscosity.

### BACKGROUND OF THE INVENTION

For the production of polysaccharides or glucans, it is possible to use fermenters in which the shear-thinning medium generated during the production is also moved in the fermenter. Such a movement can, for example, be brought about by a stirring arrangement. However, the shear-thinning media usually occurring in the production of polysaccharides or glucans have in this connection a viscosity-based property which influences the stirring process depending on a local shear stress in a fermenter.

A fluid or a medium is referred to as shear thinning when the property of the fluid shows a decreasing viscosity at high shear forces. This means: the stronger the shear acting on the fluid, the lower the viscosity/thickness. Such a fluid is also synonymously referred to as pseudoplastic. Such a decrease in viscosity upon shear stress arises, for example, through a structural change in the fluid, which structural change ensures that the individual fluid particles, for example polymer chains, can slide past each other better. Since the viscosity upon growing shear does not remain constant in a shear-thinning fluid or medium, the fluid is usually classified as a non-Newtonian fluid, meaning that the customary rudiments of flow for Newtonian fluids cannot be applied thereto. Therefore, the customary flow-related considerations of fluids no longer apply, and mixing can no longer be achieved with simple stirrer geometries.

If, then, such a shear-thinning medium is stirred, the local shear stress leads to a local reduction in viscosity, meaning that a higher flowability of the shear-thinning medium occurs locally. This requires stirrer geometries different from those for, for example, Newtonian fluids, in order to achieve by means of the stirrer geometry a uniform circulation and distribution within a fermenter volume.

Different stirrer geometries are known from the prior art. For example, “Xanthan Production in Stirred Tank Fermenters: Oxygen Transfer and Scale-up” by Holger Herbst, Adrian Schumpe and Wolf-Dieter Deckwer describes a reactor in which the diameter ratio of stirrer and stirring volume is not greater than 0.7.

“Performance of the Scaba 6SRGT Agitator in Mixing of Simulated Xanthan Gum Broths” by Enrique Galindo and Alvin W. Nienow describes, for example, a stirrer geometry which has a diameter of 0.2 m and has a diameter ratio of stirrer to stirring volume of less than 0.5.

“Mass Transfer Coefficient in Stirred Tank Reactors for Xanthan Gum Solutions” by Felix Garcia-Ochoa and Emilio Gomez describes a stirrer which is used for a 20 liter volume and has a diameter of 10 cm, the diameter of the tank being 30 cm, yielding a diameter ratio of stirrer to tank volume of 0.3.

“Oxygen Transfer and Uptake Rates during Xanthan Gum Production” by F. Garcia-Ochoa, E. Gomez Castro and V. E. Santos describes a stirrer tank geometry in which a diameter ratio of stirrer to tank diameter is 0.42.

“Effect of Mixing Behavior on Gas-Liquid Mass Transfer in Highly Viscose, Stirred Non-Newtonian Liquids” by Hans-Jürgen Henzler and Gerd Obernosterer describes a diameter ratio of stirrer to tank of not greater than 0.65.

WO 2004/058377 describes a stirrer geometry in which a baffle cylinder is provided in a tank, the stirrer extending only up to the baffle geometry in the tank volume.

EP 1 258 502 describes simple stirrer geometries for the production of an alkoxyl compound.

It has been found that all these previously described stirrer geometries for the stirring and the uniform realization of a shear-thinning medium in a fermenter are not suitable for ensuring a sufficiently uniform shear influence or for providing a sufficiently high region of low viscosity.

### SUBJECT MATTER OF THE PRESENT INVENTION

Against the background of the known prior art, it can be considered as an object to provide a uniform shear influence or a high region of low viscosity in a shear-thinning medium within a fermenter using a stirring arrangement.

This object is achieved by the subject matter of the independent claims. Advantageous developments are embodied in the dependent claims.

According to one embodiment of the invention, a fermenter for producing a shear-thinning medium is provided, the fermenter comprising: a tank volume and a stirring arrangement having a first stirring element having at least one stirring blade, a second stirring element having at least one stirring blade and a rotation axis, wherein the first stirring element and the second stirring element are fixed on the rotation axis such that they rotate with the rotation axis and are spaced axially, wherein the rotation axis when used as intended is aligned substantially parallel with respect to the direction of the earth gravitation field, and wherein the tank volume has in the region of the stirring elements substantially the shape of a circular cylinder and the rotation axis is situated substantially on the central axis of the circular cylinder, wherein the stirring blades of the first stirring element and the stirring blades of the second stirring element extend up to at least 0.8 times the distance between central axis of the circular cylinder and a wall of the circular cylinder, giving a ratio (d/D) of stirring-element diameter (d) to inner diameter (D) of the tank of at least 0.8.

In this way, it is possible to achieve within a fermenter for producing a shear-thinning medium a uniform shear influence on the shear-thinning medium and, in particular, to achieve in the shear-thinning medium a high region of low viscosity. Owing to the comparatively large diameter of the stirring element, which approaches close to the inner wall of the tank volume, it is possible to subject a large region of the shear-thinning medium to a shear stress, meaning that the viscosity in the shear-thinning medium is reduced or decreased in large regions. Owing to the arrangement of a first stirring element and of a second stirring element above/below each another, it is further possible to achieve a shear stress on the shear-thinning medium in a large region not only in the radial direction, but also in the axial direction, meaning that the viscosity decreases in a comparatively large region upon an actuation or rotation of the stirring elements with the rotation axis. It should be understood that, although the stirring elements can comprise also just a single stirring blade, the diameter of the stirring element is understood as the circle which is marked by the outmost tip of the also just single stirring blade. In this connection, it should be understood that the stirring blades can, in their radial extension direction proceeding from the rotation axis, have a uniform shape, i.e. no changing cross-sectional shape of the stirrer blade, but can also be connected to the rotation axis via rods protruding radially from the rotation axis.

According to one embodiment, a fermenter for producing an extracellular, viscosity-increasing polysaccharide is provided, which polysaccharide exhibits pseudoplastic behavior in solution, wherein the viscosity behavior of the fermentation broth produced can be described by the Ostwald de Waele power law within a shear rate range of from 1 to 150 s⁻¹ and achieves in the course of the process shear-rate-dependent minimum viscosity values which can be described by a consistency factor of K=11.98 Pas² and a flow index of n=0.1. The Ostwald de Waele power law is described in Zlokarnik, M. (2000) Dimensionsanalytische Behandlung veränderlicher Stoffgrößen [Dimensional analysis treatment of variable substance properties], in Scale-up: Modellübertragung in der Verfahrenstechnik [Scale-up: model transfer in process engineering], Wiley-VCH Verlag GmbH & Co. KGaA, Weinheim.

According to one embodiment of the invention, the first stirring element and the second stirring element is designed such that, upon a rotation of the rotation axis in a pseudoplastic medium to be stirred, a flow having a primarily axial direction ensues at the radially outer ends of the stirring blades.

In this way, the shear-thinning medium in the fermenter can be subjected to a shear stress not only in the plane of the stirring elements, but also, owing to the primarily axial conveying direction, in the volume above or below the stirring element. In this way, it is also possible for the intermediate region between the two stirring elements to be subjected to a shear stress or for the shear-thinning medium situated in said intermediate region to be brought into the shearing region of the two stirring elements. In this connection, upon the actuation of the two stirring elements, vortex-like flows can ensue within the shear-thinning medium, it being possible for the vortexes to have a larger axial extent than radial extent. Here, an axial extent means an extent parallel to the rotation axis. It should be understood that vortexing can be understood to mean not only closed flow lines, but also nonclosed flow lines or sections.

According to one embodiment of the invention, the diameter ratio of stirring elements to tank diameter d/D is 0.9±5%.

In this way, the stirring elements can approach very close to the vessel wall in order thus to achieve also in this region a high shear stress and a reduction in viscosity. As a result, the shear-thinning medium can be circulated or homogenized close to the wall region of the fermenter, resulting in the fermentation process being promoted.

According to one embodiment of the invention, the first stirring element has, in addition to the first stirring blade, a second stirring blade, wherein the first stirring blade and the second stirring blade, each with respect to the rotation axis, extend orthogonally away from the rotation axis on opposing sides of the rotation axis.

In this way, the stirring element can be designed substantially symmetrically, with two opposing stirring blades. It should be understood that it is possible too for the second stirring element and any further stirring element to have such a design. Owing to the symmetrical design of the stirring element, an uneven stress on the stirring elements and the rotation axis, especially the bearing thereof and the drive thereof, is avoided.

According to one embodiment of the invention, the first stirring element and the second stirring element have a congruent number of at least two stirring blades, wherein the stirring blades of the first stirring element are arranged offset in relation to the stirring blades of the second stirring element.

In this way, it is possible, firstly, to achieve a uniform stress on the stirring elements, the rotation axis, the bearing thereof and the drive thereof and, secondly, to achieve a more uniform shear stress on the shear-thinning medium. Owing to the offset arrangement of the stirring blades, only one stirring blade of the first and the second stirring blade always passes through a vertical plane in the tank volume at any time, meaning that a more uniform distribution of the shear-thinning medium can ensue.

According to one embodiment of the invention, the stirring blades of the first and the second stirring element are arranged offset to one another by a quarter circle.

In this way, it is possible to achieve a homogenization of the distance for a more homogeneous shear stress due to the stirring blades in the shear-thinning medium.

According to one embodiment of the invention, the stirring elements each have three or four evenly distributed stirring blades. It should be understood that, even in the case of stirring elements having three, four or more stirring blades, said stirring blades can be arranged in relation to one another such that they are offset in relation to blades of neighboring stirring elements. More particularly, they can be arranged such that one stirring blade of one stirring element is situated in a rotationally offset manner in the middle between two stirring blades of the neighboring stirring element.

According to one embodiment of the invention, the stirring surfaces of the first stirring blade and of the second stirring blade are, at least in the region of the outer ends of the stirring blades, inclined with respect to the perpendicular substantially around the extension direction of the corresponding stirring blade.

In this way, what can be achieved for example is that the shear-thinning medium is conveyed axially downward or axially upward owing to the inclined stirrer blades in the region of the outer ends, depending on in which direction with respect to the rotation direction the stirring surfaces of the stirring blades are inclined. It should be understood that not only a single stirring blade per stirring element, but also all stirring blades of the particular stirring element, can have uniformly inclined stirring surfaces.

According to one embodiment of the invention, the surfaces or stirring surfaces of the first stirring blade and of the second stirring blade are inclined with respect to the perpendicular (parallel to the rotation axis) between 30° and 60°, more particularly between 40° and 50°, more particularly by 45°±2°.

In this way, it is possible to achieve an optimum balance of a mass displacement of the shear-thinning medium during a stirring process with simultaneous shear stress.

According to one embodiment of the invention, the inclination of the stirring surfaces varies over the extension direction from the rotation axis in the direction of the tank inner walls, meaning that it is possible to achieve a uniform shear stress taking into account the different path speeds according to the distance from the rotation axis. For example, the inclination of the stirring surfaces with respect to the perpendicular can be 60° in the proximity of the rotation axis and decrease in the direction of the tips of the stirring blades down to 45°.

According to one embodiment of the invention, the fermenter further comprises an active temperature-adjustable surface for heating and/or cooling, wherein the flow profile is guided along the temperature-adjustable surface.

In this way, the fermentation process within the fermenter can be controlled and, depending on the requirement for the fermentation process, sped up or slowed down, specifically by appropriate heating or cooling of the active temperature-adjustable surface of the fermenter. In this connection, the temperature-adjustable surfaces can be provided on the tank wall, but can also be arranged within the tank volume.

According to one embodiment of the invention, the temperature-adjustable surface is formed by circumferential pipe sections which are, with respect to the rotation axis, arranged in groups in the axial direction, wherein one group extends between two stirring elements lying immediately one above another.

In this way, it is possible to achieve an efficient temperature adjustment, especially since, as a result of an axial movement of the shear-thinning medium during a stirring process, the shear-thinning medium can be moved along the temperature-adjustable surfaces or the groups of pipe sections.

According to one embodiment of the invention, the tank volume has in the region of the stirring elements substantially the shape of a circular cylinder, wherein inwardly protruding baffles can be provided in the circular cylinder, wherein the baffles extend further inward than the stirring blades extend outward in the direction of the wall of the tank volume.

In this way, there is a radial overlap of the baffles with the stirring blades, meaning that it is possible to prevent the entire volume of the shear-thinning medium from moving in a uniform rotating movement with the stirring elements, the result being that the shear stress would decrease. The inwardly protruding baffles slow down such a rotating movement of the shear-thinning medium, meaning that the shear stress is increased again and, in this way, the viscosity also decreases, the result being that the mixing of the shear-thinning medium increases again. In this connection, baffles are understood to mean structures which interrupt, redirect or very generally disrupt a generated flow. In the above-described case, a circular flow corresponding to the rotating movement of the stirring elements is interrupted or disrupted, meaning that the shear stress in the shear-thinning medium increases.

According to one embodiment of the invention, the baffles keep the pipe sections spaced away from a wall of the tank volume, wherein the pipe sections are arranged further inward in the tank volume than the stirring blades extend outward in the direction of the wall of the tank volume.

In this way, it can be ensured that the axially moving shear-thinning medium, especially at the end of the stirring blades, moves along the pipe sections of the temperature-adjustable surface and, in this way, is adjusted in temperature.

According to one embodiment of the invention, the stirring arrangement additionally has a third stirring element, a fourth stirring element and a fifth stirring element which are arranged on the rotation axis such that they are spaced apart from one another, wherein each of the stirring elements has two stirring blades which are offset by a quarter circle with respect to the stirring blades of a neighboring stirring element on the rotation axis.

In this way, it is for example possible to provide a stirring arrangement having five or more stirring elements which, for example, are fixed on the rotation axis at equal intervals and rotate with said rotation axis. In this connection, the individual stirring elements can also have three, four or more stirring blades, the result being that the offset corresponds to the half angle between two neighboring stirring blades of a stirring element. Especially in the case of three or more stirring blades per stirring element, it is possible for the stirring blades of neighboring stirring elements to also be arranged above one another, i.e., not offset in relation to one another. Owing to such a multilevel stirrer configuration, it is possible to achieve a uniform mixing of and shear stress on a shear-thinning medium even in the case of relatively large tank volumes of from 10 m³ to 1000 m³ or more.

According to one embodiment of the invention, four groups of pipe sections are provided among the five stirring elements, wherein, in each case, one group of pipe sections is arranged between two stirring elements lying immediately one above another.

In this way, it is possible to achieve a uniform temperature adjustment in the tank volume of the fermenter.

According to one embodiment of the invention, the fermenter comprises a gas supply device, the mouth of which is arranged below the at least two stirring elements.

In this way, it is for example possible to introduce oxygen in order to promote the fermentation, or to introduce a different gas in order, for example, to displace an oxygen in the shear-thinning medium. The mouths of the gas supply device can, in particular, be arranged below the coverage circle of the stirring blades. It should be understood that a further gas supply device can also be provided above the two stirring elements; in particular, a gas supply device can also be provided between two arbitrary stirring elements.

According to one embodiment of the invention, at least three pipe sections are arranged in the axial direction in a cross-sectional plane of a baffle.

This gives rises to an axially extended temperature-adjustable surface. In particular, it is possible in a cross-sectional plane of a baffle to arrange two pipe sections in the radial direction and four to five pipe sections in the axial direction. However, it should be understood that it is possible to provide an arbitrary number of pipe sections arranged radially next to one another and an arbitrary number of pipe sections arranged axially next to one another, so long as this group of pipe sections does not restrict the movement of the stirring elements.

According to one embodiment of the invention, a method for producing a polysaccharide using an above-described fermenter is provided. The above-described features based on a device are also applicable, mutatis mutandis, to a corresponding method.

According to one embodiment of the invention, the polysaccharide in solution exhibits pseudoplastic behavior, wherein the viscosity behavior of a produced fermentation broth is described by the Ostwald de Waele power law within a shear rate range of from 1 to 150 s⁻¹. wherein the fermentation broth produced by the method achieves in the course of the process shear-rate-dependent minimum viscosity values which are characterized by a consistency factor of K=11.98 Pas² and a flow index of n=0.1.

According to one embodiment of the invention, the polysaccharide is an extracellular, viscosity-increasing polysaccharide.

According to one embodiment of the invention, the polysaccharide is a glucan, which encompasses in particular at least one of an α-glucan, a β-glucan and a xanthan gum, or is substantially an α-glucan, a β-glucan or a xanthan gum.

The individual above-described features can self-evidently also be combined with one another, the result being that in some cases advantageous interactions going beyond the sum of the individual effects may also ensue.

These aspects and other aspects of the present invention will be elucidated and illustrated by reference to the exemplary embodiments described hereinbelow.

### BRIEF DESCRIPTION OF THE DRAWINGS

Exemplary embodiments are described below with reference to the following drawings.

FIG. 1 shows a sectional view through a fermenter according to one exemplary embodiment of the invention.

FIG. 2 shows one detail from a stirring arrangement according to one exemplary embodiment of the invention.

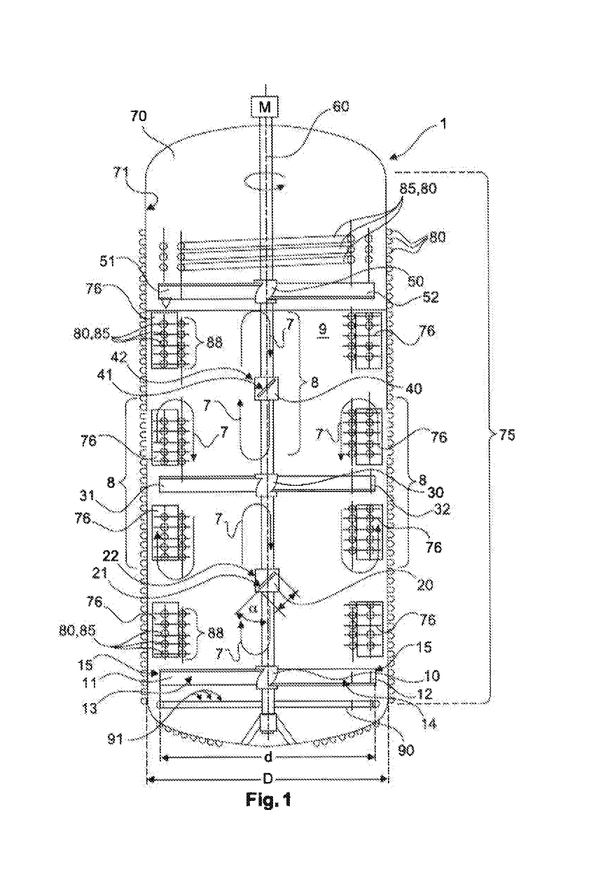

FIG. 1 shows a fermenter according to one exemplary embodiment of the invention for producing a shear-thinning medium. In this connection, the fermenter 1 has a tank volume 70, which is defined by a wall of the tank volume 71. Situated in the tank volume 70 is a stirring arrangement having multiple stirring elements 10, 20, 30, 40, 50, which are each fixed on a rotation axis 60 and can rotate together with the rotation axis 60, driven via a motor M, around the rotation axis 60. The stirring elements in the embodiment shown in FIG. 1 each have two stirring blades, a first stirring blade 11 and a second stirring blade 12 for the first stirring element 10, and also analogously for the second, third, fourth and fifth stirring element 20, 30, 40, 50 a respectively first stirring blade 21, 31, 41, 51 and a second stirring blade 22, 32, 42, 52. The two stirring blades of a stirring element extend from the central axis or the rotation axis 60 in the direction of the wall 71 of the tank volume 70. In the embodiment shown in FIG. 1, each stirring element has two stirring blades which have substantially a constant inclination over the extension direction. Each stirring blade 11, 12 has a correspondingly inclined surface 13, 14, by means of which the shear-thinning medium is, upon a rotation of the rotation axis 60, substantially conveyed in an axial direction, i.e., with a component parallel to the rotation axis. In this connection, during the conveyance of the shear-thinning medium, said medium can be pushed either upward or downward by the inclined surfaces 13, 14, depending on in which direction the rotation axis 60 with the stirring elements 10 to 50 fixed thereto rotates. For example, in this connection, a flow direction 7 which may be vortex-like ensues, wherein this flow has an axial component which is stronger than a radial component. The vortex or the vortex-like flow is depicted in a simplified manner by arrows with the flow direction 7. However, in reality, the flow profile will be substantially more complex, especially since there is a differing exertion of force on the medium to be stirred 9, the shear-thinning medium, owing to the differing path speed according to the distance from the rotation axis 60. In the arrangement shown in FIG. 1, the vortexes 8 substantially form such that there is an axial circulation of the medium to be stirred 9, meaning that the regions between the stirring elements 10 to 50 are also subjected to a movement and are circulated such that they also reach the shearing region of the stirring blades of the stirring elements. In the embodiment shown in FIG. 1, each stirring element has two stirrer blades which are each arranged offset in relation to the stirrer blades of a stirring element arranged immediately adjacently, For instance, the stirring blades of the lowest stirring element 10, of the middle stirring element 30 and of the top stirring element 50 extend laterally in the image plane, whereas the stirring blades of the intermediate stirring elements 20 and 40 extend forward from the image plane or backward into the image plane.

The stirring blades of the stirring elements, as shown in FIG. 1, have an inclination which is substantially constant over the extension direction, in this case with the angle α, which specifies the inclination with respect to the perpendicular, i.e., the extension direction of the rotation axis 60. It should be understood that the inclination of the stirring blades can change over the extension length of the stirring blades from the rotation axis 60 up to the blade tip, meaning that it is possible to take into account the differing path speed of the stirring elements according to the distance from the rotation axis 60. In particular, the inclination of the surfaces with respect to the direction of the rotation axis 60 can be greater in the region close to the axis than in the region far from the axis. In this connection, it should be understood that, in the case of a greater inclination, the axial propulsion component is lower than in the case of a smaller inclination.

In the embodiment shown in FIG. 1, the filling level in the tank volume 70 is situated just below the uppermost stirring element, meaning that the stirring element 50 in FIG. 1 is arranged above the medium to be stirred 9. Below the lowest stirring element 10, there is provided a gas supply device, the mouth of which is below the lowest stirring element 10. In this connection, the mouths 91 can be below the coverage circle of the two stirring blades 11, 12 of the first stirring element 10. When gas is introduced by means of the gas supply device 90 into the medium to be stirred 9, the volume of the medium to be stirred increases by the introduced gas bubbles. Consequently, the fill level in the tank volume rises, meaning that the fill level in this case can rise to above the uppermost stirring element 50, meaning that the uppermost stirring element 50 contributes to the stirring process. Depending on in which direction the rotation axis 60 with the stirring elements 10 to 50 fixed thereto rotates, the rise of the gas bubbles in the medium to be stirred 9 is promoted, specifically when the stirrer blades press upward the medium to be stirred 9 because of the inclined surfaces of the stirring blades, or slowed down, when the stirring blades move the medium downward when the rotation axis 60 rotates in the opposite direction and the stirring surfaces push downward the gas bubbles in the medium to be stirred 9.

The stirring blades 11, 21, 31, 41, 51; 12, 22, 32, 42, 52 extend from the rotation axis 60 to just before the wall 71 of the tank volume 70. The diameter of the stirring elements, which is to be understood in the context of the invention to mean the diameter of the scan circle of the particular stirring element, is approximately as large as the diameter of the tank volume 70 in the region of a circle-cylinder-based cross-sectional section of the tank volume 75.

The diameter ratio between the diameter of the stirring elements d to the diameter of the tank volume D is, for example, 0.9. It should be understood that the diameter ratio d/D can be selected as large as possible, meaning that a stirring movement of the stirring elements 10 to 50, said movement taking place up into the edge region of the tank volume, brings about at these points a shear stress on the shear-thinning medium, meaning that a good mixing of the medium to be stirred 9 is achieved there. The diameter ratio d/D can, for example, be up to 0.99, provided it is ensured that the radially outer ends 15 of the stirring blades do not collide with the wall 71 of the tank volume.

To support the fermentation process in the fermenter 1, it is possible to provide temperature-adjustable surfaces 80 which can adjust the temperature of the tank volume 70 or the medium to be stirred 9 situated therein. Said temperature-adjustable surfaces can, for example, be arranged in the form of outer cooling coils on the outside of the tank volume 70. Alternatively or additionally, it is also possible to arrange within the tank volume 70 temperature-adjustable surfaces which are, for example, then situated between the stirring elements. The temperature-adjustable surfaces provided in the tank volume 70 can, for example, be circumferential pipe sections 85 which can, for example, be arranged in the form of spiral pipes in the tank volume 70. In this connection, the circumferential pipe sections can be provided both spirally and circularly, it being possible to provide the spiral arrangement for a sequential flow-through. However, it is also possible to provide circular pipe sections which are either subjected to a flow-through in parallel, or which can be subjected to a flow-through in a sequential manner through an appropriate bend at right angles and a connection between a pipe section and an overlying pipe section through the bend at right angles. In the embodiment shown in FIG. 1, there are provided between the stirring elements groups 88 of circumferential pipe sections, which generally consist of two pipe sections lying next to one another in the radial direction, and five pipe sections arranged below/above one another. Such a group 88 of pipe sections can be subjected to a flow-through of a temperature-adjusting agent, either a cooling agent or a heating agent, in a sequential manner through an appropriate spiral guide. Owing to the design of the stirring blades and the resulting, preferably axial, flow of the medium to be stirred 9 within the tank volume, an overflow on the temperature-adjustable surfaces 80 or on the groups 88 of circumferential pipe sections 85 is achieved, meaning that it is possible in this region to achieve a temperature adjustment of the medium to be stirred 9. The fermentation process can be controlled by means of the temperature adjustment.

To prevent the rotation of the stirring elements 10 to 50 from moving the medium to be stirred in one entire rotating movement, meaning that the medium to be stirred substantially no longer moves with respect to the stirring elements, it is possible to provide baffles 76 in the tank volume 70. Said baffles can, for example, be paddles or plates which extend inwardly from the wall 71 of the tank volume 70, for example in the direction of the rotation axis. It should be understood that the baffles 76 can also extend into the tank volume 70 in a vertically and/or horizontally inclined manner and need not necessarily point toward the rotation axis 60. The baffles can be immediately fixed to the wall 71 of the tank volume 70 or else protrude into the tank volume 70 through spacers. In this connection, the baffles overlap radially with the stirring blades of the stirring elements, meaning that there is a radial overlap of baffles 76 and stirring blades 11, 21, 31, 41, 51, etc. In this way, a rotation movement of the medium to be stirred 9 is interrupted, or disrupted, and the relative movement of the stirring blades with respect to the medium to be stirred 9 is thus ensured. Consequently, it is possible to maintain by means of the stirring elements a shear stress on the medium to be stirred 9, the result being that the medium to be stirred is diluted and better flowable at this point.

In this connection, the baffles 76 can also serve as holding structures for the temperature-adjustable surfaces. In particular, the baffles can serve as holding structures for the groups of circumferential pipe sections and position them. In this connection, both the baffles 76 and the groups 88 of pipe sections 85 can extend at any distance into the space between the stirring elements, so long as they do not restrict or impede the rotation of the stirring elements around the rotation axis 60.

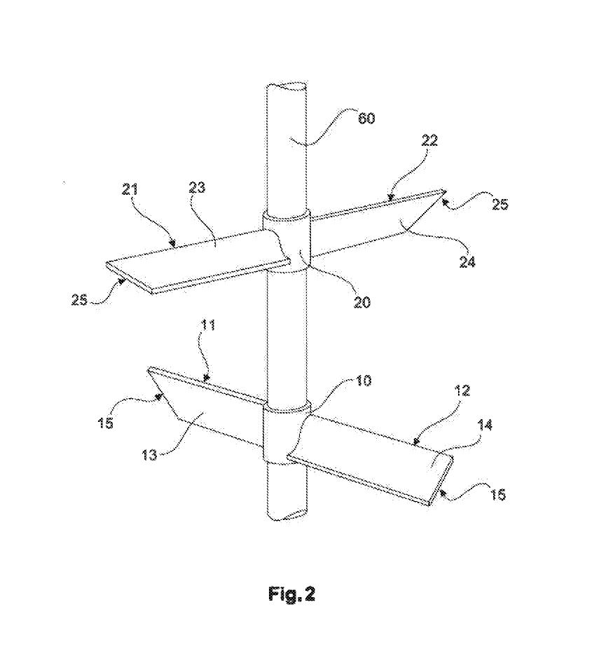

FIG. 2 shows a detail from a stirring arrangement which is constructed from the rotation axis 60 and a first stirring element 10 and a second stirring element 20. It should be understood that further stirring elements above and below the first or second stirring element are not ruled out here. In this connection, each of the two stirring elements 10, 20 has a first stirring blade 11 or 21 and a second stirring blade 12 or 22. In the arrangement shown in FIG. 2, the stirring blades are inclined by about 45° with respect to the extension direction of the rotation axis 60. As a result, the surfaces 13 and 14 and 23 and 24 are inclined and can, depending on the rotation direction, speed up in either an upward or downward direction the medium to be stirred 9. Owing to the applied shear forces, the shear-thinning medium becomes thinner and thus more flowable, meaning that mixing is improved. In this connection, the outer ends 15 and 25 extend to just before the wall 71 of the tank volume 70, which, however, is not shown in FIG. 2.

In FIG. 2, although the two stirring elements 10, 20 each have two stirring blades extending away on opposing sides, the stirring elements 10, 20 can also have three, four or more stirring blades. In this connection, said stirring blades can be distributed evenly along the circumference, meaning that a substantially symmetrical stirring element is provided.

In FIG. 2, the stirring blades of the first stirring element 11, 12 are arranged offset with respect to the stirring blades of the second stirring element 21, 22. FIG. 2 depicts, in particular, an offset by a quarter circle. However, it should be understood that the offset can also vary in size, meaning that, for example, in the case of three existing stirring elements on the rotation axis, the offset of neighboring stirring elements can be 60° in each case, meaning that a continued offset from stirring element to stirring element is a further 60° in each case.

Especially if more than two stirring blades are provided in the case of one stirring element or multiple stirring elements, the stirring blades can also be arranged above one another, i.e., without an offset in the case of neighboring stirring elements.

It should be noted that the present invention can be used in particular also for shear-thinning media which can serve for the extraction of petroleum, for example xanthan gum, glucans, more particularly α- and β-glucans.

### LIST OF REFERENCE SIGNS

1 Fermenter; stirrer for shear-thinning media for a fermentation process
7 Flow direction
8 Vortex
9 Medium to be stirred
10 First stirring element
11 First stirring blade of the first stirring element
12 Second stirring blade of the first stirring element
13 Inclined surface of the first stirring blade of the first stirring element
14 Inclined surface of the second stirring blade of the first stirring element
15 Radially outer end of the stirring blades of the first stirring element
20 Second stirring element
21 First stirring blade of the second stirring element
22 Second stirring blade of the second stirring element
23 Inclined surface of the first stirring blade of the second stirring element
24 Inclined surface of the second stirring blade of the second stirring element
25 Radially outer end of the stirring blades of the second stirring element
30 Third stirring element
31 First stirring blade of the third stirring element
32 Second stirring blade of the third stirring element
40 Fourth stirring element
41 First stirring blade of the fourth stirring element
42 Second stirring blade of the fourth stirring element
50 Fifth stirring element
51 First stirring blade of the fifth stirring element
52 Second stirring blade of the fifth stirring element
60 Rotation axis of the stirring arrangement
70 Tank volume
71 Wall of the tank volume
75 Section of the tank which has the shape of a circular cylinder
76 Baffles; holding structure for circumferential pipe sections
80 Temperature-adjustable surface for heating and/or cooling
85 Circumferential pipe sections
88 Group of circumferential pipe sections
90 Gas/oxygen supply
91 Mouth of the gas/oxygen supply
α (alpha) Inclination angle of the stirring blades with respect to the perpendicular
d Outer diameter of the stirring elements
D Inner diameter of the tank volume in the region 75

1.-16. (canceled)

17. A fermenter for producing a shear-thinning medium, comprising:
a tank volume (70) and
a stirring arrangement having a first stirring element (10) having at least one stirring blade (11), a second stirring element (20) having at least one stirring blade (21), and a rotation axis (60),
wherein the first stirring element (10) and the second stirring element (20) are fixed on the rotation axis (60) such that they rotate with the rotation axis and are spaced axially,
wherein the rotation axis (60) when used as intended is aligned substantially parallel with respect to the direction of the earth gravitation field, and
wherein the tank volume (70) has in the region of the stirring elements (10, 20) substantially the shape of a circular cylinder (75) and the rotation axis (60) is situated substantially on the central axis of the circular cylinder (75), wherein the stirring blades (11, 12) of the first stirring element (10) and the stirring blades (21, 22) of the second stirring element (20) extend up to at least 0.8 times the distance between central axis of the circular cylinder (75) and a wall (71) of the circular cylinder, giving a ratio (d/D) of stirring-element diameter (d) to inner diameter (D) of the tank of at least 0.8,
further comprising an active temperature-adjustable surface (80) for heating and/or cooling, wherein the flow profile (7) is guided along the temperature-adjustable surface,
and wherein the temperature-adjustable surface (80) is formed as circumferential pipe sections (85) which are, with respect to the rotation axis (60), arranged in groups (88) in the axial direction, wherein one group extends between two stirring elements (10, 20) lying immediately one above another.

18. The fermenter according to claim 17, wherein the first stirring element (10) has, in addition to the first stirring blade (11), a second stirring blade (12), wherein the first stirring blade and the second stirring blade, with respect to the rotation axis (60), extend orthogonally away from the rotation axis on opposing sides of the rotation axis.

19. The fermenter according to claim 17, wherein the first stirring element (10) and the second stirring element (20) have a congruent number of at least two stirring blades (11, 12; 21, 22), wherein the stirring blades (11, 12) of the first stirring element are arranged offset in relation to the stirring blades (21, 22) of the second stirring element.

20. The fermenter according to claim 17, wherein the stirring blades (11, 12; 21, 22) of the first and the second stirring element (10, 20) are arranged offset to one another by a quarter circle.

21. The fermenter according to claim 17, wherein stirring surfaces (13, 14) of the first stirring blade (11) and of the second stirring blade (12) are, at least in the region of the outer ends of the stirring blades, inclined with respect to the perpendicular substantially around the extension direction of the corresponding stirring blade.

22. The fermenter according to claim 21, wherein the stirring surfaces (13, 14) of the first stirring blade (11) and of the second stirring blade (12) are inclined with respect to the perpendicular between 30° and 60°.

23. The fermenter according to claim 21, wherein the stirring surfaces (13, 14) of the first stirring blade (11) and of the second stirring blade (12) are inclined with respect to the perpendicular between 40° and 50°.

24. The fermenter according to claim 21, wherein the stirring surfaces (13, 14) of the first stirring blade (11) and of the second stirring blade (12) are inclined with respect to the perpendicular is 45°+/−2°.

25. The fermenter according to claim 17, wherein the tank volume (70) has in the region of the stirring elements (10, 20, 30, 40, 50) substantially the shape of a circular cylinder (75), wherein inwardly protruding baffles (76) are provided in the circular cylinder, wherein the baffles extend further inward than the stirring blades (11, 12, 21, 22) extend outward in the direction of the wall (71) of the tank volume (70).

26. The fermenter according to claim 25, wherein the baffles keep the pipe sections (85) spaced away from a wall (71) of the tank volume, wherein the pipe sections are arranged further inward in the tank volume (70) than the stirring blades (11, 12, 21, 22) extend outward in the direction of the wall (71) of the tank volume (70).

27. The fermenter according to claim 25, wherein at least one pipe section (85) and at least three pipe sections (85) are arranged in the radial direction and in the axial direction, respectively, in a cross-sectional plane of a baffle (76).

28. The fermenter according to claim 17, wherein the stirring arrangement additionally has a third stirring element (30), a fourth stirring element (40) and a fifth stirring element (50) which are arranged on the rotation axis (60) such that they are spaced apart from one another, wherein each of the stirring elements (30, 50) has two stirring blades (31, 32; 51, 52) which are offset by a quarter circle with respect to the stirring blades (21, 22; 41, 42) of a neighboring stirring element (20, 40) on the rotation axis (60).

29. The fermenter according to claim 28, wherein four groups (88) of pipe sections (85) are provided among the five stirring elements, wherein, in each case, one group (88) of pipe sections (85) is arranged between two stirring elements (10, 20; 20, 30; 30, 40; 40, 50) lying immediately one above another.

30. The fermenter according to claim 17, further comprising a gas supply device (90), the mouth (91) of which is arranged below the at least two stirring elements (10, 20, 30, 40, 50).

31. A method for producing a polysaccharide comprising using the fermenter according to claim 17.

32. The method according to claim 31, wherein the polysaccharide in solution exhibits pseudoplastic behavior, wherein the viscosity behavior of a produced fermentation broth is described by the Ostwald de Waele power law within a shear rate range of from 1 to 150 s⁻¹, wherein the fermentation broth produced by the method achieves in the course of the process shear-rate-dependent minimum viscosity values which are characterized by a consistency factor of K=11.98 Pas² and a flow index of n=0.1.

33. The method according to claim 31, wherein the polysaccharide is an extracellular, viscosity-increasing polysaccharide.

34. The method according to claim 31, wherein the polysaccharide is a glucan.

35. The method according to claim 31, wherein the polysaccharide is an α-glucan, a β-glucan or a xanthan gum.
